(19) United States
(12) Patent Application Publication
(10) Pub. No.: US 2019/0246978 A1
(43) Pub. Date: Aug. 15, 2019

(54) System and Method for Echogenically Enhancing Nerve Fibers Using Targeted Metallic Particles

(71) Applicant: Avent, Inc., Alpharetta, GA (US)

(72) Inventors: Justin J. Coker, Laguna Niguel, CA (US); Kenneth C. Hsu, Tustin, CA (US)

(21) Appl. No.: 16/335,859

(22) PCT Filed: Oct. 11, 2016
(86) PCT No.: PCT/US2016/056340

§ 371 Date: Mar. 22, 2019

(51) Int. Cl. A61B 5/00; A61B 5/055; A61B 6/00; A61B 6/03; A61B 8/08; A61K 49/22

(52) U.S. Cl. CPC A61B 5/4893; A61B 5/055; A61B 6/506; A61B 6/481; A61M 5/007; A61B 8/085; A61B 8/481; A61K 49/222; A61B 6/032

(57) ABSTRACT

The present invention is directed to a system and method for echogenically enhancing a target site within a patient during a medical procedure. The system includes an imaging system having a display for viewing the target site, a plurality of metallic particles configured to selectively target and bind to one or more locations at the target site, and a delivery mechanism for delivering the plurality of metallic particles into the patient towards the target site. As such, the method includes delivering, via the delivery mechanism, the plurality of metallic particles into the patient and allowing the metallic particles to selectively target and bind to the target site. The method also includes viewing, via a display of an imaging system, the target site with the plurality of metallic particles bound thereto.

### FIELD OF THE INVENTION

The present invention relates generally to the field of medical imaging, and more particularly, to a system and method for echogenically enhancing nerve fibers using targeted metallic particles.

### BACKGROUND

Conventional imaging technologies such as CT, MRI, and ultrasound can be categorized as structural imaging modalities. Such imaging modalities are generally able to identify anatomical structures but are not without drawbacks. For example, certain imaging modalities are not particularly helpful during nerve block procedures, as the technologies have previously not been efficient at delivering clear images of the nerve block anatomy, surrounding structures, and/or the needle location.

Recent advancements in imaging modalities, however, have provided for effective nerve block procedures to be performed using such imaging. For example, in medical imaging, selective particles have been shown to be able to target certain cell types, such as cancer cells and/or nerve bundles. More specifically, magnetic materials and/or magnetic particles are often employed in the body to enhance image contrast of such cells. The magnetic nanoparticles can be passivated by biocompatible coatings such as dextrin, citrate, olystyrene, and/or divinylbenzene. These coatings can also detoxify the particles, resulting in enhanced lifetimes in vivo. Such targeted particles have shown promise in enhancing imaging of such cells using imaging modalities.

Thus, developments in structural imaging modalities that continuously improve upon medical imaging during various medical procedures, such as nerve block procedures, would be welcomed in the art. More specifically, a system and method for echogenically enhancing nerve fibers or bundles using targeted metallic particles would be advantageous.

### SUMMARY OF THE INVENTION

Objects and advantages of the invention will be set forth in part in the following description, or may be obvious from the description, or may be learned through practice of the invention.

In one aspect, the present invention is directed to a system for echogenically enhancing a target site within a patient during a medical procedure. The system includes an imaging system having a display for viewing the target site, a plurality of metallic particles configured to selectively target and bind to one or more locations at the target site, and a delivery mechanism for delivering the plurality of metallic particles into the patient towards the target site.

In one embodiment, the metallic particles may be selected based on their chemical or atomic structure being attracted to one or more locations at the target site. In another embodiment, the metallic particles may include metallic nanoparticles. For example, in certain embodiments, each of the metallic nanoparticles may have a diameter of from about 1 nanometer to about 100 nanometers. In further embodiments, each of the metallic particles may include any suitable metallic materials that are biocompatible in the human body, including but not limited to silver, gold, copper, or combinations thereof.

In another embodiment, a quantity of the plurality of metallic particles may be from about one thousand (1,000) to about one million (1,000,000) metallic particles. In further embodiments, any number of metallic particles may delivered into the body of the patient, including less than 1,000 particles or more than 1,000,000 particles, e.g. depending on the medical procedure and/or the target site being echogenically enhanced.

In additional embodiments, the plurality of metallic particles are configured to temporarily bind to the one or more locations at the target site for a predetermined dwell time before diffusing into the patient. More specifically, in such embodiments, the predetermined dwell time of the plurality of metallic particles may include from about one day to about two days. In further embodiments, any suitable dwell time may be sufficient for temporarily binding the metallic particles to the target site and then diffusing into the body, including less than one day or more than two days.

In particular embodiments, the delivery mechanism of the system may include at least one of a needle, a syringe, or combinations thereof. In further embodiments, the target site of the patient may include nerve cells, cancer cells, nerve sheaths, nerve bundles, nerve fibers, or any other site within the patient. In addition, the medical procedure may include a peripheral nerve block procedure. Thus, in certain embodiments, the metallic particles are configured to selectively target and bind to nerve fibers during a nerve block procedure.

In yet another embodiment, the imaging system may include a CT scanner, an MRI scanner, an ultrasound imaging system, or any other imaging system than can benefit from the echogenically-enhanced particles as described herein.

In another aspect, the present invention is directed to a method for echogenically enhancing a target site of a patient during a medical procedure. The method includes delivering, via a delivery mechanism, a plurality of metallic particles into the patient. Further, the method includes allowing the plurality of metallic particles to selectively target and bind to the target site. The method also includes viewing, via a display of an imaging system, the target site with the plurality of metallic particles bound thereto.

In one embodiment, the method also includes adjusting a quantity of the metallic particles being delivered into the patient as a function of the medical procedure. In another embodiment, the method may include selecting one or more of the plurality of metallic particles based on their chemical or atomic structure being attracted to the target site. It should also be understood that the plurality of metallic particles may have any of the additional properties and/or features as described herein.

In yet another aspect, the present invention is directed to a method for echogenically enhancing a target site of a patient during a medical procedure. The method includes delivering, via a delivery mechanism, a plurality of high-density particles into the patient. For example, in certain embodiments, the high-density particles may have a density of from about 1800 kilograms per cubic centimeter (kg/cm³) to about 22,000 kg/cm³. Further, the method includes allowing the plurality of high-density particles to selectively target and bind to the target site. The method also includes viewing, via a display of an imaging system, the target site with the plurality of high-density particles bound thereto. It should be understood that the method may further include any of the additional method steps/or features as described herein.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a schematic diagram of one embodiment of an ultrasound imaging system according to the present disclosure;

FIG. 2 illustrates a schematic diagram of one embodiment of suitable components that may be included in a processor of the ultrasound imaging system of FIG. 1;

FIG. 3 illustrates a schematic diagram of one embodiment of an ultrasound probe configured on a patient's skin so as to generate an image of a target site of the patient according to the present disclosure;

FIG. 4 illustrates a schematic diagram of one embodiment of an ultrasound probe configured on a patient's skin so as to generate an image of a target site of a patient according to the present disclosure, particularly illustrating a delivery mechanism delivering a plurality of targeted particles into the patient towards the target site;

FIG. 5 illustrates a schematic diagram of one embodiment of an ultrasound probe configured on a patient's skin so as to generate an image of a target site of a patient according to the present disclosure, particularly illustrating a plurality of targeted particles bound to a plurality of nerve fibers at a target site of the patient; and

FIG. 6 illustrates a flow diagram of one embodiment of a method for echogenically enhancing a target site of a patient during a medical procedure according to the present disclosure.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

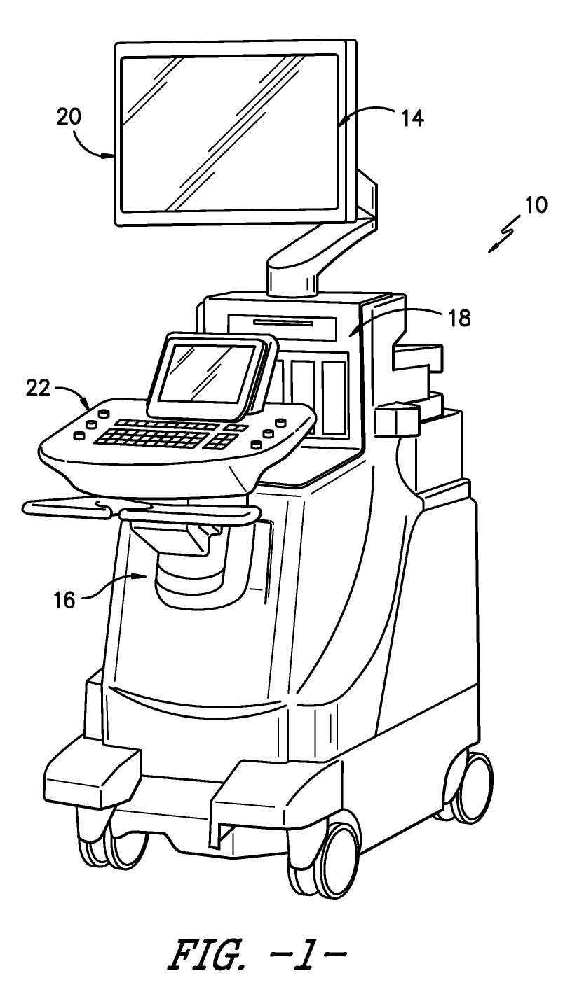

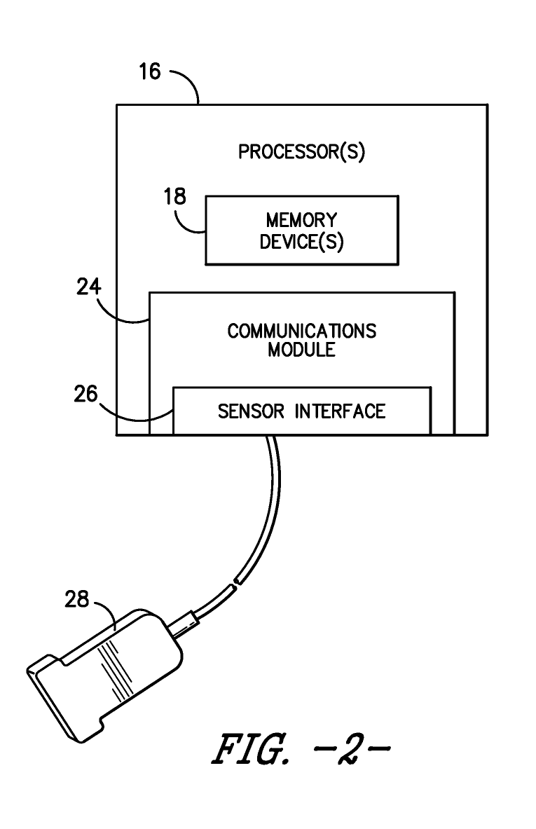

Referring now to the drawings, FIGS. 1 and 2 illustrate one embodiment of an imaging system 10 and an associated processor 16 of the imaging system 10, respectively, according to the present disclosure. As described herein, the imaging system 10 may correspond to an ultrasound imaging system (as shown), a computer tomography (CT) scanner, a magnetic resonance imaging (MRI) scanner, or any other imaging system than can benefit from the echogenically-enhanced particles as described herein. Further, as shown, the imaging system 10 generally includes one or more processor(s) 16 and associated memory device(s) 18 configured to perform a variety of computer-implemented functions (e.g., performing the methods and the like and storing relevant data as disclosed herein), as well as a user display 20. In addition, the imaging system 10 may include a user interface 22, such as a computer and/or keyboard, configured to assist a user in generating and/or manipulating an image 14 displayed by the user display 20.

Additionally, as shown in FIG. 2, the processor(s) 16 may also include a communications module 24 to facilitate communications between the processor(s) 16 and the various components of the imaging system 10, e.g. any of the components of FIG. 1. Further, the communications module 24 may include a sensor interface 26 (e.g., one or more analog-to-digital converters) to permit signals transmitted from one or more probes (e.g. the ultrasound probe 28) to be converted into signals that can be understood and processed by the processor(s) 16. It should be appreciated that the ultrasound probe 28 may be communicatively coupled to the communications module 24 using any suitable means. For example, as shown in FIG. 2, the ultrasound probe 28 may be coupled to the sensor interface 26 via a wired connection. However, in other embodiments, the ultrasound probe 28 may be coupled to the sensor interface 26 via a wireless connection, such as by using any suitable wireless communications protocol known in the art. As such, the processor(s) 16 may be configured to receive one or more signals from the ultrasound probe 28.

As used herein, the term “processor” refers not only to integrated circuits referred to in the art as being included in a computer, but also refers to a controller, a microcontroller, a microcomputer, a programmable logic controller (PLC), an application specific integrated circuit, a field-programmable gate array (FPGA), and other programmable circuits. The processor(s) 16 is also configured to compute advanced control algorithms and communicate to a variety of Ethernet or serial-based protocols (Modbus, OPC, CAN, etc.). Furthermore, in certain embodiments, the processor(s) 16 may communicate with a server through the Internet for cloud computing in order to reduce the computation time and burden on the local device. Additionally, the memory device(s) 18 may generally comprise memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) 18 may generally be configured to store suitable computer-readable instructions that, when implemented by the processor(s) 16, configure the processor(s) 16 to perform the various functions as described herein.

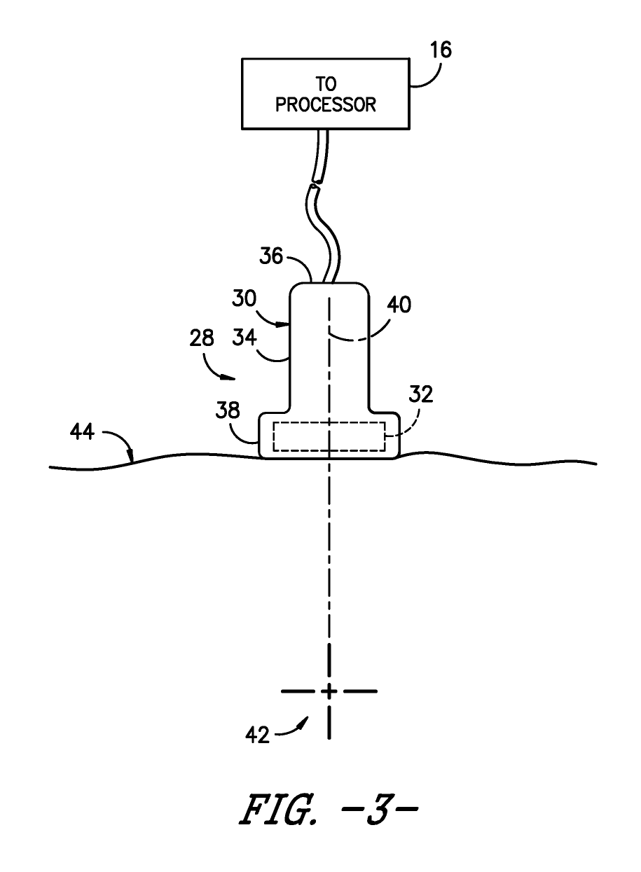

Thus, as shown in FIGS. 2 and 3, the ultrasound probe 28 may include a transducer housing 30 and a transducer transmitter 32 mounted therein. As is generally understood, the transducer transmitter 32 is configured to emit and/or receive ultrasound beams. For example, as shown, the transducer housing 30 includes a body 34 extending from a proximal end 36 to a distal end 38 along a longitudinal axis 40. Further, the distal end 38 of the body 34 includes an internal cavity (not numbered). As such, the transducer transmitter 32 may be configured within the internal cavity so as to scan a target site 42 within a patient when the ultrasound probe 28 is placed on the patient's skin 44 during a medical procedure. As described herein, the target site 42 of the patient may include any anatomical structure and/or surrounding tissue within the human body, including but not limited to nerve cells, cancer cells, nerve sheaths, nerve bundles, nerve fibers, or any other site within the patient. As such, in certain embodiments, the medical procedure may include a peripheral nerve block procedure. An image of the target site 42 can then be generated and displayed to a user via the display 20 of the ultrasound imaging system 10 (FIG. 1).

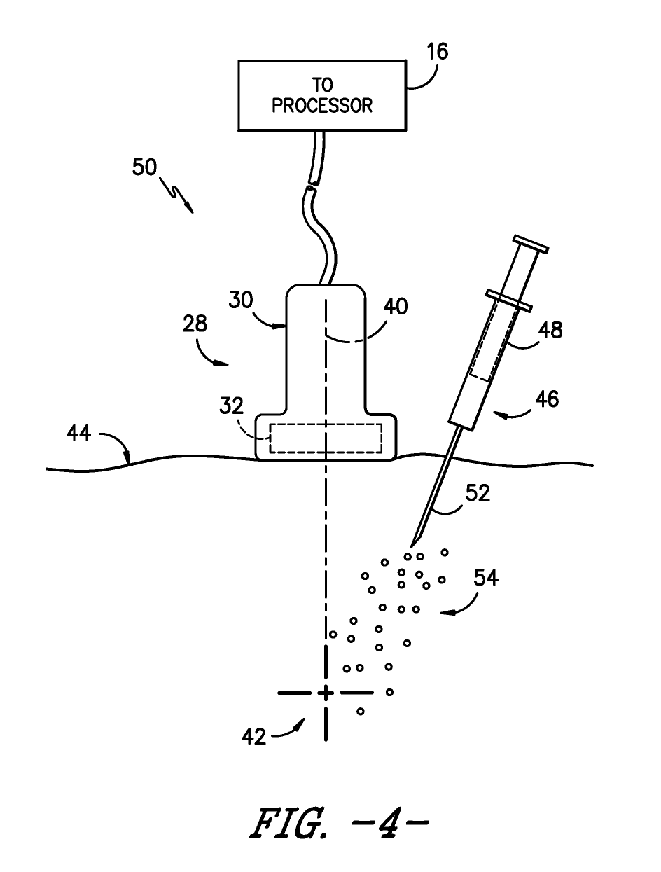

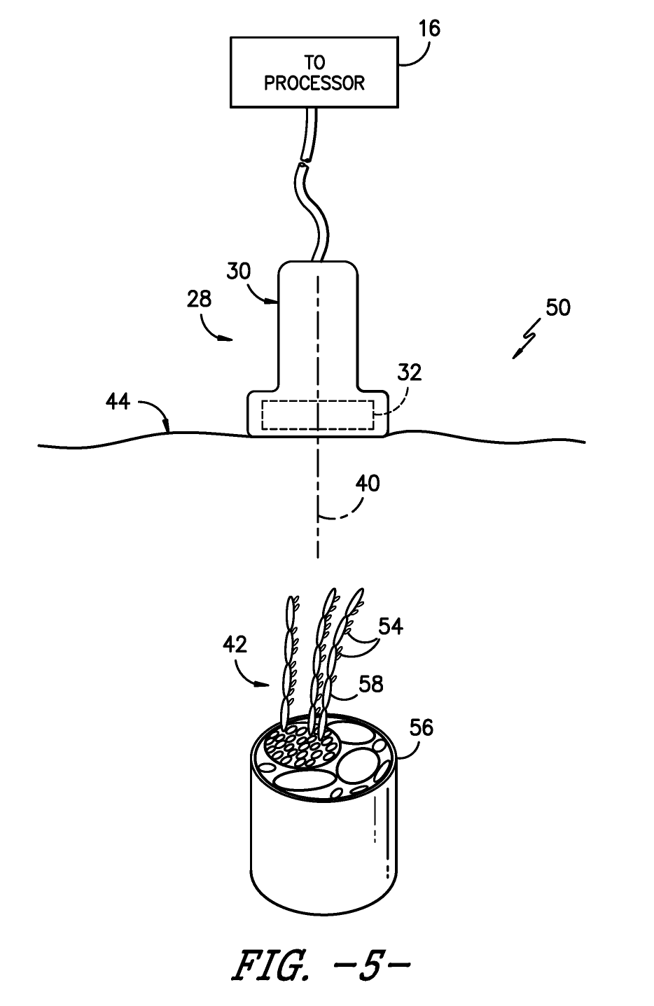

Referring now to FIGS. 4 and 5, schematic diagrams of various embodiments of a system 50 for echogenically enhancing the target site 42 of a patient during a medical procedure are illustrated. As shown, the system 50 may include an imaging system, e.g. the imaging system 10 of FIG. 1 which includes the user display 20 for viewing the target site 42. Further, the system 50 includes a plurality of particles 54 configured to selectively target and bind to one or more locations at the target site 42. In one embodiment, the particles 54 may include high-density particles so as to echogenically enhance the target site 42 when viewed via the imaging system 10. More specifically, in certain embodiments, the high-density particles 54 may have a density of from about 1800 kilograms per cubic centimeter (kg/cm³) to about 22,000 kg/cm3. Further, in particular embodiments, the high-density particles 54 may include metallic particles. For example, in certain embodiments, the metallic particles 54 may be constructed of any suitable metallic materials that are biocompatible with the human body, including but not limited to silver, gold, copper, or combinations thereof.

More specifically, the metallic particles 54 may be selected based on their chemical or atomic structure being attracted to one or more locations at the target site 42. For example, as shown in FIG. 5, the target site 42 of the patient may include a nerve bundle 56 having a plurality of nerve fibers 58. Thus, as shown, the metallic particles 54 are configured to selectively target and bind to one or more of the nerve fibers 58 during a nerve block procedure. In such embodiments, when the metallic particles 54 are injected into the patient, the particles 54 are attracted to the nerve fibers 58 at the target site 42 and will easily bind thereto or form bonds therewith. It should be understood that the plurality of metallic particles 54 temporarily bind to the one or more locations at the target site 42 for a predetermined dwell time before diffusing into the patient as the human body naturally tries to remove any foreign materials that are present therein. As such, in certain embodiments, the predetermined dwell time of the particles 54 may include from about one day to about two days. In further embodiments, any suitable dwell time may be sufficient for binding the metallic particles 54 to the target site 42 and then diffusing into the body, including less than one day or more than two days. For example, in one embodiment, the dwell time may correspond to a predetermined number of hours substantially corresponding to the length of the medical procedure.

In additional embodiments, the metallic particles 54 as described herein may have any suitable size. For example, in certain embodiments, the particles 54 may correspond to nanoparticles. As used herein, the term ‘nanoparticles’ generally refers to extremely small particles that have a diameter of from about 1 nanometer to about 100 nanometers.

It should be understood that any suitable quantity of the particles 54 may be injected into the patient so as to enhance the echogenic properties of the target site 42. For example, in certain embodiments, from about one thousand (1,000) to about one million (1,000,000) of the particles 54 may be injected or delivered into the patient and can be determined based on the procedure and/or the anatomical structure or surrounding tissue of the target site 42. In additional embodiments, any number of the particles 54 may delivered into the patient, including less than 1,000 particles or more than 1,000,000 particles, e.g. depending on the medical procedure and/or the properties of the target site 42.

Still referring to FIG. 4, the system 50 also includes a delivery mechanism 46 for delivering the high-density particles 54 into the patient towards the target site 42. For example, as shown in the illustrated embodiment, the delivery mechanism 46 may include a syringe 48 configured with a needle 52, a needle-guide assembly, or any other suitable delivery mechanism. For example, in certain embodiments, where the delivery mechanism 46 corresponds to a needle guide assembly, the assembly may include, at least, a needle and a catheter. As such, it should be understood that the needle as well as the catheter of the needle guide assembly can be inserted into the patient in any particular order or simultaneously so as to deliver the particles 54 described herein. For example, in one embodiment, the needle guide assembly may include an over-the-needle (OTN) catheter assembly in which the catheter is coaxially mounted over the needle. Alternatively, the needle may be mounted over the catheter. In such embodiments, the needle may act as an introducer such that it places the catheter within the patient to deliver the particles 54 and is later removed.

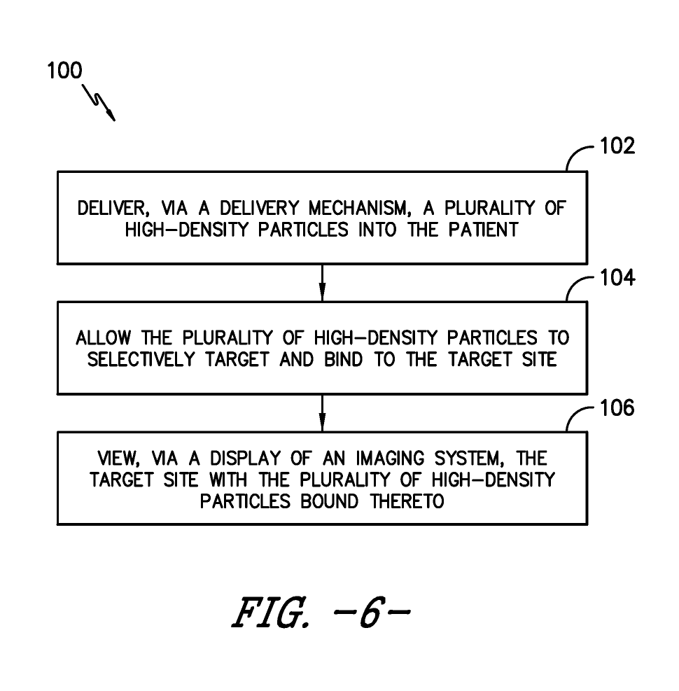

Referring now to FIG. 6, a flow diagram of one embodiment of a method 100 for echogenically enhancing a target site 42 of a patient during a medical procedure is illustrated. As shown at 102, the method 100 includes delivering, via the delivery mechanism 46, a plurality of high-density particles 54 into the patient, i.e. towards the target site 42. Further, as shown at 104, the method 100 includes allowing the plurality of high-density particles 54 to selectively target and bind to the target site 42. As shown at 106, the method 100 includes viewing, e.g. via the user display 20 of the imaging system 10, the target site 42 with the plurality of high-density particles 54 bound thereto (as shown in FIG. 5). As such, the target site 42 is echogenically enhanced via the high-density particles 54 and can be easily viewed under ultrasound or any other imaging system.

In one embodiment, the method 100 may also include adjusting a quantity of the particles 54 being delivered into the patient as a function of the medical procedure. In another embodiment, the method 100 may include selecting one or more of the plurality of particles 54 based on their chemical or atomic structure being attracted to the target site 42.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

1. A system for echogenically enhancing a target site of a patient during a medical procedure, the system comprising:
an imaging system comprising, at least, a display for viewing the target site;
a plurality of metallic particles configured to selectively target and bind to one or more locations at the target site; and,
a delivery mechanism for delivering the plurality of metallic particles into the patient towards the target site.

2. The system of claim 1, wherein the metallic particles are selected based on their chemical structure being attracted to one or more locations at the target site

3. The system of claim 1, wherein the plurality of metallic particles comprise metallic nanoparticles.

4. The system of claim 3, wherein each of the metallic nanoparticles comprises a diameter of from about 1 nanometer to about 100 nanometers.

5. The system of claim 1, wherein each of the plurality of metallic particles comprises at least one of the following metallic materials: silver, gold, copper, or combinations thereof.

6. The system of claim 1, wherein a quantity of the plurality of metallic particles comprises from about one thousand (1,000) to about one million (1,000,000) metallic particles.

7. The system of claim 1, wherein the plurality of metallic particles temporarily bind to the one or more locations at the target site for a predetermined dwell time before diffusing into the patient.

8. The system of claim 7, wherein the predetermined dwell time of the plurality of metallic particles comprises from about one day to about two days.

9. The system of claim 1, wherein the delivery mechanism comprises at least one of a needle or a syringe.

10. The system of claim 1, wherein the target site of the patient comprises at least one of nerve cells, cancer cells, nerve sheaths, nerve bundles, or nerve fibers.

11. The system of claim 1, wherein the imaging system comprises at least one of a CT scanner, an MRI scanner, or an ultrasound imaging system.

12. The system of claim 1, wherein the medical procedure comprises a peripheral nerve block procedure.

13. A method for echogenically enhancing a target site of a patient during a medical procedure, the method comprising:
delivering, via a delivery mechanism, a plurality of metallic particles into the patient;
allowing the plurality of metallic particles to selectively target and bind to the target site; and,
viewing, via a display of an imaging system, the target site with the plurality of metallic particles bound thereto.

14. The method of claim 13, further comprising adjusting a quantity of the metallic particles being delivered into the patient as a function of the medical procedure.

15. The method of claim 13, further comprising selecting one or more of the plurality of metallic particles based on their chemical structure being attracted to the target site.

16. The method of claim 13, wherein the plurality of metallic particles comprise metallic nanoparticles, wherein each of the metallic nanoparticles comprises a diameter of from about 1 nanometer to about 100 nanometers.

17. The method of claim 13, wherein each of the plurality of metallic particles comprises at least one of the following metallic materials: silver, gold, copper, or combinations thereof.

18. The method of claim 13, wherein the plurality of metallic particles temporarily bind to one or more locations at the target site for a predetermined dwell time before diffusing into the patient, wherein the predetermined dwell time of the metallic particles comprises from about one day to about two days.

19. The method of claim 13, wherein the target site of the patient comprises at least one of nerve cells, cancer cells, nerve sheaths, nerve bundles, or nerve fibers.

20. A method for echogenically enhancing a target site of a patient during a medical procedure, the method comprising:
delivering, via a delivery mechanism, a plurality of high-density particles into the patient, the high-density particles comprises a density of from about 1800 kilograms per cubic centimeter (kg/cm³) to about 22,000 kg/cm³;
allowing the plurality of high-density particles to selectively target and bind to the target site; and,
viewing, via a display of an imaging system, the target site with the plurality of high-density particles bound thereto.
